(19) United States
(12) Patent Application Publication
(10) Pub. No.: US 2024/0093139 A1
(43) Pub. Date: Mar. 21, 2024

(54) NATURAL GROWN FIBER COMPOSITES FOR SUSTAINABLE BUILDING MATERIALS

(71) Applicant: CASE WESTERN RESERVE UNIVERSITY, Cleveland, OH (US)

(72) Inventors: Xiong Yu, Beachwood, OH (US); Xijin Zhang, Cleveland, OH (US)

(21) Appl. No.: 18/467,960

(22) Filed: Sep. 15, 2023

Related U.S. Application Data
(60) Provisional application No. 63/375,732, filed on Sep. 15, 2022.

(51) Int. Cl. C12N 1/14; E04B 1/78

(52) U.S. Cl. CPC C12N 1/14; E04B 1/78

(57) ABSTRACT

A mycelium composite material includes a fungi generated mycelium fiber matrix and a biofiller (organic or inorganic or combined) dispersed within the matrix. The biofiller provides a structural element and nutrition source for the fungi and the mycelium fibers bind the biofiller.

### RELATED APPLICATION

This application claims priority from U.S. Provisional Application No. 63/375,732 filed Sep. 15, 2022, the subject matter of which is incorporated herein by reference in its entirety.

### GOVERNMENT FUNDING

This invention was made with government support under 1563238 awarded by the National Science Foundation. The government has certain rights in the invention.

### BACKGROUND

In light of its important role in addressing environmental issues and achieving economic benefits, energy saving has received increasing attention in the recent years. The heating and cooling energy used for building operations account for almost one third of the total energy consumption in the world. Thermal insulation materials are utilized in the building envelope to improve the energy efficiency, by reducing the thermal loads both in the hot and cold climate zones. The insulation layer provides a border between the indoor and outside environment, ensuring the thermal comfort of the inhabitants and reduces energy usage in operating the building.

Synthetic or petroleum-derived materials with good thermal performance, such as glass wool, expanded polystyrene (EPS), and polyurethane (PU) foam etc., are widely used as building insulation materials. However, their manufacturing processes incur carbon footprints. In addition, some of the synthetic or petroleum-derived materials may contain harmful substances, limiting their application for building construction due to possible health concerns. Furthermore, most common types of synthetic foams are not biodegradable and lead to generation of large amount of waste at the end of their service life. In other words, synthetic insulation materials have negative impacts regarding their manufacturing, application, and recycling, causing damage to the environment and to human health. Therefore, the need for sustainable buildings calls for the development of eco-friendly insulation materials.

### SUMMARY

Embodiments described herein relate to a naturally grown composite technology that utilizes fungi to produce mycelium fiber and form composite materials for sustainable building applications. The material system includes bio-filler, which provides structural element as well as nutrition source and fungi spore. Fungi utilize the bio-source to produce fibers that bond the filler materials.

The outcome is to naturally grow mycelium-composite that can be used for building insulation and other applications. The resultant mycelium composites include a core of colonized substrate, encased by a layer of water-repellent fungal skin. The microstructure of mycelium composites include fungal fibers grown from various feedstocks that bond adjacent feedstocks to form the composite materials. The materials also feature excellent fire resistance.

The fiber-reinforced composite can be produced using organic or inorganic fillers from various waste sources (e.g., yard waste, agriculture residual, and other bio based feedstock). The natural grown composite material provides a sustainable way to produce building materials.

### BRIEF DESCRIPTION OF THE DRAWINGS

FIGS. 1(A-E) illustrate a schematic of preparation process of mycelium composites detailing the main stages. (A) Incubation of fungal spawn, (B) Transferring myceliated feedstock to molds, (C) Growing substrate for 4 days, (D) Flipping over sample to grow for another 4 days, (E) Harvesting mycelium composite after drying.

FIGS. 2(A-B) illustrate SEM images of mycelium composite (A) fungal mycelium grown from grains, (B) fungal mycelium bridge between two grains.

FIGS. 3(A-H) illustrate EDS analysis of mycelium composite.

FIG. 4 illustrates water absorption in fungal skin and mycelium composites when subjected to different relative humidity.

FIGS. 5(A-D) illustrate load-displacement curves of mycelium composites grown with different amount feedstock tested under (A) low, (B) middle, and (C) high RH, as well as (D) flexural strength obtained from three-point bending tests.

FIG. 6 illustrates a schematic of changes occur in composites when exposed to low, middle, and high RH (low RH leading to brittle rye berry grain, middle RH improves the ductility of rye berry grain, high RH compromises the bonding between mycelium fiber and rye berry grain).

FIGS. 7(A-C) illustrates representative crack patterns of mycelium composites subjected to 3-point pending test after exposure to environment with (A) low, (B) middle, and (C) high RH.

FIG. 8 illustrates prototypical office model and multi-layer envelope.

FIG. 9 illustrates a schematic of cross-section of experimental setup.

FIG. 10 illustrates the development of measured temperature processes at each layer in MC 45 g-1.

FIG. 11 illustrates a schematic of cross-section of experimental setup to measure the heat capacity of the mycelium composite brick.

FIGS. 12(A-B) Inner roof surface profiles in the building model with different insulators in Albuquerque, NM on: (A) February 9th; (B) July 4th.

FIGS. 13(A-B) comparison of annual energy use of building with LECA, EV insulator and with mycelium-composite (MC) insulator at different climate conditions: (A) cooling energy; (B) heating energy.

FIGS. 14(A-H) illustrate monthly energy consumption of building with common types of insulation versus with the new mycelium-composite insulation at different cities: (A) Miami, FL; (B) Phoenix, AZ; (C) San Francisco, CA; (D) Albuquerque, NM; (E) Salt Lake, UT; (F) Helena, MT; (G) Duluth, MN; (H) Fairbanks, AK.

FIGS. 15(A-B) illustrate comparison of the performance of different insulators (mycelium-composite insulator, LECA, EV insulators) for building operation under different climate conditions (A) annual energy cost, and (B) annual equivalent carbon emission.

### DETAILED DESCRIPTION

Embodiments described herein relate to a naturally grown composite technology that utilizes fungi to produce mycelium fiber and form composite materials for sustainable building applications. The material system includes bio-filler which provide structural element as well as nutrition source and fungi spore. Fungi utilize the bio-source to produce fibers that bond the filler materials.

The outcome is to naturally grow mycelium-composite that can be used for building insulation and other applications. The resultant mycelium composites includes a core of colonized substrate, encased by a layer of water-repellent fungal skin. The microstructure of mycelium composites include fungal fibers grown from various feedstocks that bond adjacent feedstocks to form the composite materials. The materials also feature excellent fire resistance.

The fiber-reinforced composite can be produced using organic or inorganic fillers from various waste sources (e.g., yard waste, agriculture residual, and other bio based feedstock). The natural grown composite material provides a sustainable way to produce building materials.

Other embodiments described herein relate to a mycelium composite material includes a fungi generated mycelium fiber matrix and a biofiller (organic or inorganic or combined) dispersed within the matrix. The biofiller can provide a structural element and nutrition source for the fungi, and the mycelium fibers can bind the biofiller. The mycelium composite material can be used as a building insulation material in, for example, a building envelope.

In some embodiments, the building insulation material can include a mycelium composite material that includes a core of fungi colonized substrate encased by a water-repellant fungi skin. The core and skin can include a mycelium fiber network.

Still other embodiments described herein relate to a building envelope that includes a mycelium composite insulation provided on or within the building envelope. The mycelium composite insulation can include a fungi generated porous mycelium fiber matrix and a biofiller dispersed within the matrix. The biofiller provides a structural element and nutrition source for the fungi, and the mycelium fibers can bind the biofiller.

Yet other embodiments described herein relate to method of forming a building material. The method includes cultivating a mycelium generating fungus on a biomaterial feedstock. The cultivated fungus and feedstock are ground after generation of fungal spores and/or fungal hyphae colonization into the feedstock. The ground fungi and feedstock are then transferred to a mold. The ground fungi and feedstock are cultivated in the mold such that a mycelium composite is formed with a core of fungi colonized substrate encased by a water-repellant fungi skin.

In some embodiments, during cultivation, nutrition or minerals can be provided to the mycelium composite to tailor fungi properties (e.g., adding source of silica in the nutrition source to enhance fire resistance).

In some embodiments, the mycelium composite can be heated after cultivation to kill any living microbes in the mycelium composite.

In other embodiments, the mycelium composite can be compressed or pressure molded to enhance mechanical strength.

Still other embodiments relate to a method of insulating a building envelope. The method includes providing a mycelium composite insulation on or within the building envelope. The mycelium composite insulation includes a fungi generated porous mycelium fiber matrix and a biofiller dispersed within the matrix. The biofiller can provide a structural element and nutrition source for the fungi, and the mycelium fibers can bind the biofiller.

### Example

We investigated the mechanical and thermal properties of mycelium composite under a variety of environmental conditions. The energy saving performance of mycelium-composite insulation for buildings under different climate zones is evaluated using Energy Plus, an industry standard for building energy performance assessment. Mycelium-composite insulation was produced with an edible fungus (Pleurotus ostreatus) that was cultivated in rye berries feedstock. The microstructure and chemical elements mapping of grown mycelium composite were studied by Scanning Electron Microscope/Energy Dispersion Spectroscopy (SEM/EDS). The mechanical behaviors and failure modes of mycelium composites with different density were investigated after exposure to different levels of relative humidity. EnergyPlus was utilized to conduct the energy usage simulations based on the experimental measured results of the physical and thermal properties of mycelium composites. Comparative analysis was conducted on building models specifically between using mycelium-composite insulation, which is naturally derived, and traditional natural insulation materials, i.e., lightweight expanded clay aggregate (LECA) and expanded vermiculite (EV). In addition to comparing materials, this study uses these building models to illustrate the impact of different climate zones on energy consumption. The results of both simulations indicate that the mycelium composite performs better than traditional natural insulation materials under different climate conditions, with the exception of the very hot climate zone. Mycelium composite insulation is a new and innovative form of natural insulation whose energy performance compared favorably to the traditional insulation materials. It therefore presents a more environmental benign and sustainable building insulation option.

### Model and Methodology

The building energy assessment was simulated by EnergyPlus version 8.9.0, which is a flagship software developed by the Lawrence Berkeley National Lab of the US Department of Energy. The performance of EnergyPlus has been validated with the experimental results and other widely used energy simulation software. It includes three major components, i.e., a simulation manager, a heat and mass balance simulation module, and a building system simulation module. Thermal loads in a building (with a user-defined geometry, envelope, and HVAC schedule) can be calculated by the heat and mass balance simulation module. Then the building system simulation module allows the calculation of corresponding heating and cooling energy response under given thermal loads by a certain climate condition. Since a number of variables are required for EnergyPlus analyses, a list of symbols used is summarized in Table 1.

TABLE 1
List of symbols.

| | | | |
|---|---|---|---|
| d | Thickness (m) | τₛ | Solar transmittance |
| | | | at normal incidence |
| Rₐ | Roughness | τᵥ | Visible transmittance |
| | | | at normal |
| ρ | Density (kg/m³) | R_{sf} | Incidence Front side |
| | | | solar reflectance at |
| | | | normal incidence |
| k | Thermal conductivity | R_{sb} | Back side solar |
| | (W/(m · K)) | | reflectance at |
| | | | normal incidence |
| C | Specific heat capacity | R_{vf} | Front side visible |
| | (J/(kg · K)) | | reflectance at |
| | | | normal incidence |
| αₜ | Thermal absorptance | R_{vb} | Back side visible |
| | | | reflectance at |
| | | | normal incidence |
| αₛ | Solar absorptance | ε | Emissivity |
| αᵥ | Visible absorptance | | |

### Building Model

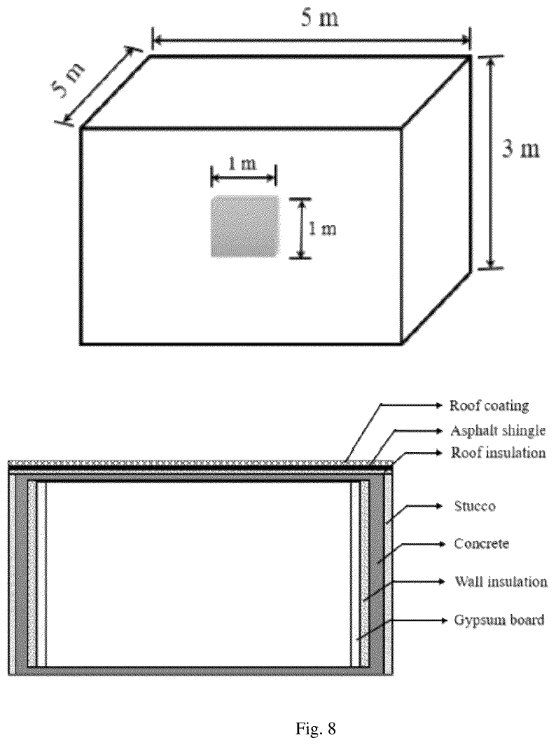

The prototype office building is assumed to have a square floor plan (5 m×5 m) and a height of 3 m. In the simulation for a building, a typical multi-layer envelope was chosen. Transparent windows with a dimension of 1 m×1 m were located at the center of both the northern and southern walls. Other parts of the wall were considered opaque and all the walls were exposed to the outside environment. The geometry and material layers of building envelope structure are provided in FIG. 8 and Table 2. For example, the roof included a roof coating layer, asphalt shingle layer, and roof insulation layer above the concrete structure. The walls included a stucco exterior layer, concrete, wall insulation and gypsum board interior layers. The indoor temperature was controlled by a building HVAC system to keep a comfortable environment. The operational conditions of the building, including the indoor temperature range, ventilation, occupancy, etc., used in the computational simulations are summarized in Table 3.

TABLE 2
Constituents of building envelope and properties of the components

| | | | | | C | | | |
|---|---|---|---|---|---|---|---|---|
| | d (m) | Rₐ | p (kg/m³) | k (W/(m · K)) | (J/(kg · K)) | aₜ | aₛ | aᵥ |
| Roof | | | | | | | | |
| Roof | 0.01 | Smooth | 1121 | 0.16 | 1460 | 0.9 | 0.94 | 0.96 |
| coating | | | | | | | | |
| Asphalt | 0.015 | Smooth | 2500 | 1.5 | 2100 | 0.9 | 0.94 | 0.96 |
| shingle | | | | | | | | |
| Concrete | 0.05 | Medium | 2242.6 | 1.7296 | 836.8 | 0.9 | 0.65 | 0.65 |
| | | rough | | | | | | |
| Wall | | | | | | | | |
| Stucco | 0.005 | Smooth | 1858.1 | 0.6918 | 836.8 | 0.9 | 0.92 | 0.92 |
| Concrete | 0.2 | Medium | 2242.6 | 1.7296 | 836.8 | 0.9 | 0.65 | 0.65 |
| | | rough | | | | | | |
| Gypsum | 0.01 | Smooth | 784.9 | 0.16 | 830 | 0.9 | 0.4 | 0.4 |
| board | | | | | | | | |
| Floor | | | | | | | | |
| Concrete | 0.1 | Medium | 2242.6 | 1.7296 | 836.8 | 0.9 | 0.65 | 0.65 |
| | | rough | | | | | | |
| Window | D (m) | τₛ | τᵥ | k (W/(m · K)) | Rsf | Rsb | Rvf | Rvb |
| Glass | 0.01 | 0.23 | 0.25 | 2.1073 | 0.72 | 0 | 0.7 | 0 |

[symbol]

TABLE 3
The operational conditions of the building systems.
HVAC system

| Heating set-point | 20° | C. | Heating resource: |
|---|---|---|---|
| temperature | | | natural gas |
| Cooling set-point | 26° | C. | Cooling resource: |
| temperature | | | electricity |
| Ventilation rate | 0.203 | m²/ [symbol] | |
| Total heating | Auto-sized to | | |
| capacity | design day | | |
| Total cooling | Auto-sized to | | |
| capacity | design day | | |
| Lighting system | | | |
| Lighting load | 1600 | W | Fully dimmable |
| | | | [0%-100%] |
| Other | | | |
| People occupancy | 8 | | |
| Equipment load | 1200 | W | Weekdays |
| | | | [8:00-24:00] |
| Ground temperature | 20° | C. | |
| Time step | 10 | min | |

| | |
|---|---|
| Simulation time | One year |

[symbol]

indicates data missing or illegible when filed

TABLE 4
Density measurement of mycelium-composite brick

| | | | | | |
|---|---|---|---|---|---|
| Sample | Substrate | Mass | Mass | Volume | Density |
| ID | (g) | (g) | (g) | (cm³) | (kg/m³) |

| | | | | |
|---|---|---|---|---|
| MC 45 g-1 | 15.703 | 15.808 | 26.400 | 599 |

| | | |
|---|---|---|
| 1 MC 45 g-2 | 45 (split into | 15.713 |
| 2 MC 45 g-3- | three samples) | 16.008 |

### Characteristics of the Mycelium Composite Insulation Layer

Properties of materials used for building envelope are obtained from the recommended values. Since mycelium composite are a unique building material with unique structures, its properties are custom measured. The procedures and results are summarized below.

### Density of Mycelium Composite Brick

The mass of mycelium composites ranged from 15.703 g to 16.008 g. Volume of the composite was measured as 26.400±0.085 cm³ based on the Archimedes' principle. The average density of three duplicates of the mycelium composites was 599 kg/m3 shown in Table 4. While other non-pressed mycelium-composite materials have a lower density of 100-220 kg/m³, because the feedstock of rye berries used in this study is more dense than other feedstocks such as cotton and sawdust which have been utilized in other studies.

### Thermal Conductivity

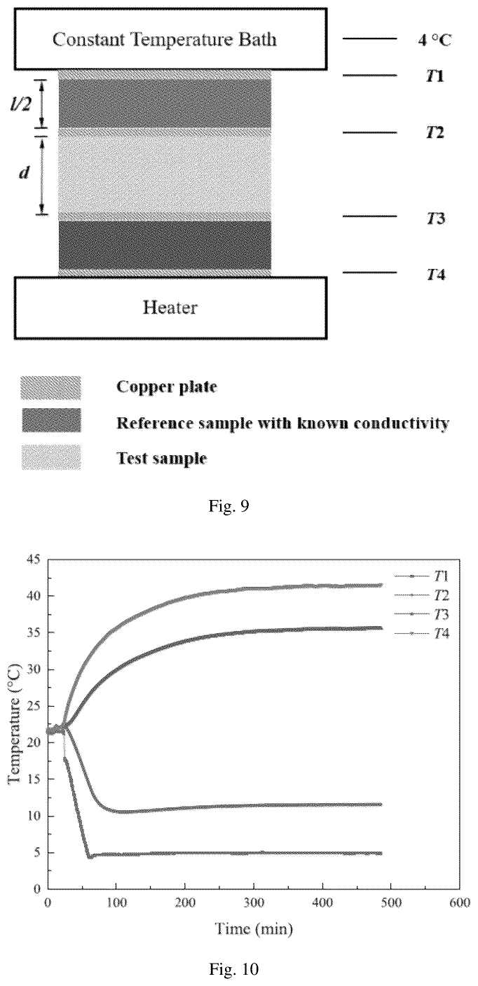

The divided bar method was employed to measure the thermal conductivity of mycelium-composite brick. An axial heat flow was produced by the apparatus shown in FIG. 9. It includes a heater as a heat source at the bottom and a constant temperature bath at the top. The test sample was connected in serial to reference samples via copper plates. Temperature sensors were installed in the layer of copper plates. When thermal equilibrium was reached, the temperature difference at various copper plates revealed a close relationship with thermal conductivity of materials. The thermal resistance of copper plates was assumed negligible in this study. Acrylic glass with known thermal conductivity of 0.17 W/(m·K) acted as a reference sample. The thermal conductivity of the test sample was solved by Eqs. (1)-(4) based on the continuity of heat flux across the reference samples and the test sample. Both the reference samples and the test samples were placed at room temperature and humidity for 24 h prior to test. The ambient relative humidity and temperature were measured as 49±1.2% and 24±0.75° C.

d Kₛ/K=ΔT2

ΔT₁+ΔT3 (1)

ΔT₁=T₂−T₁ (2)

ΔT₂=T₃−T₂ (3)

ΔT₃=T₄−T₃ (4)
where K is the thermal conductivity of test sample, d is the thickness of test sample, Kₛ is the thermal conductivity of reference sample, l is the total thickness including those of the two layers of reference samples, ΔT1J ΔT2J ΔT3 are the temperature difference between two copper plates.

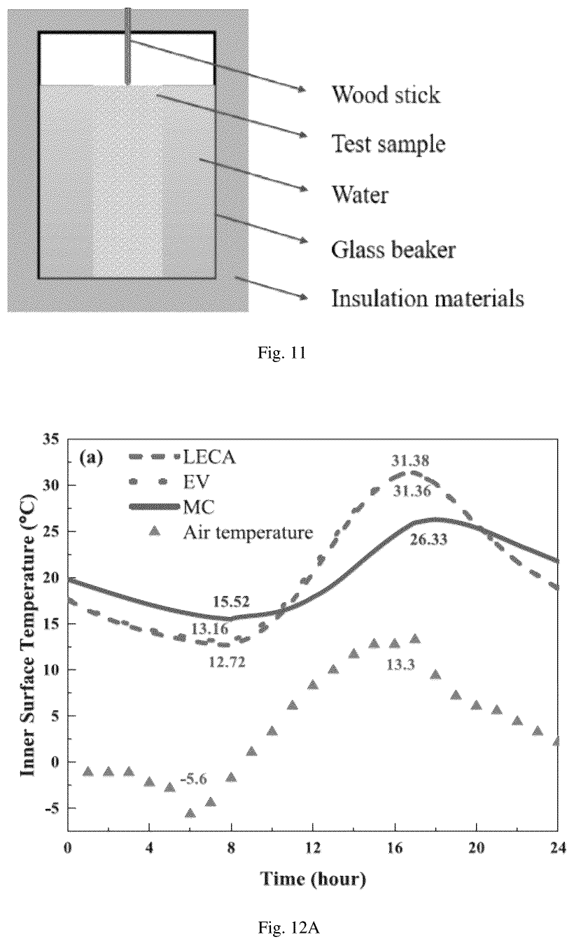

The measured temperature changes of each copper layer in MC 45 g-1 are shown in FIG. 10 and the equilibrium temperatures (T1, T2, T3 and T4) and thermal conductivity of each composite are described in Table 5 (see FIG. 11).

### Specific Heat Capacity

The specific heat capacity is the amount of heat required to raise the temperature of material per unit mass by 1° C. It is also an important indicator of the sensitivity of the material to the applied thermal load. Samples were placed in the oven for 24 h to keep the initial temperature at 60° C. It allowed heat exchange when the hot sample was placed in the cold water and glass chamber. The temperature in the system would finally reach an equilibrium. A wooden stick was used to ensure that test samples were fully submerged in the water since the sample is lighter than water. A thick layer of insulation materials was wrapped around the chamber to eliminate the heat exchange with the ambient environment. Assuming perfect thermal insulation, the magnitude of heat loss in a warmer body is the same as the heat gained in the cooler body. The thermal sensors were placed into water and on the chamber to measure the time-dependent temperature and obtain the final equilibrium temperature. The experimental data and results of three independent measurements of the specific heat capacity are shown in Table 6. The average specific heat capacity of MC 45 g is 6894±120 J/(kg·K).

### Data for Common Types of Insulation Materials

The lightweight expanded clay aggregate (LECA) and the expanded vermiculite (EV) have low density and good thermal insulation properties. They are natural insulation materials for building construction. Therefore, comparative simulation was performed in the building models with mycelium-composite and LECA, EV that served as roof and wall insulation layers. The energy consumption in the building with mycelium-composite insulation was evaluated and compared with the results in buildings with LECA and EV insulations. The data on the properties of common insulation material are summarized in Table 7.

### Geographic Locations in Different Climate Zones

The energy consumption of buildings with mycelium-composite insulation material or conventional (LECA and EV) insulation materials were simulated for different US climate zones to assess their relative performance. 8 cities, i.e., Miami, FL; Phoenix, AZ; San Francisco, CA; Albuquerque, NM; Salt Lake, UT; Helena, MT; Duluth, MN; Fairbanks, AK, were selected to represent all ASHRAE US climate zones with different thermal criteria. Table 8 lists the thermal conditions of 8 representative locations identified in ASHRAE Standard 90.1-2010 in the United States (Briggs et al., 2003). Their weather data was inputted into the EnergyPlus models through the climate database by the U.S. Department of Energy (E and EnergyPlus Energy).

### Analysis of Energy Performance

The model of buildings with mycelium-composite insulation or with conventional insulations (LECA, EV) was established in the 8 U.S. climate zones. First, to illustrate the general observation, the daily temperature on the inner roof surface was evaluated in the building with mycelium-composite insulation or conventional insulations located in Albuquerque, NM during the heating or cooling seasons. Then, buildings with different insulators under various climate thermal conditions were modeled and their annual and monthly energy costs were compared. Finally, the annual energy cost and carbon emission of building models with different insulation materials were analyzed and compared.

TABLE 7
Input data of insulation material

| | | | | | C | | | | |
|---|---|---|---|---|---|---|---|---|---|
| Insulators | d (m) | Rₐ | p (kg/m³) | k (W/(m · K)) | (J/(kg · K)) | aₜ | aₛ | aᵥ | Refer |
| LECA | Roof/0.025 | Medium | 520 | 0.097 | 950 | 0.9 | 0.7 | 0.7 | (Gauvin and |
| | | rough | | | | | | | Vette, 2020; |
| | | | | | | | | | Crawley et al., |
| | | | | | | | | | 2001) |
| | Wall/0.05 | | | | | | | | |
| EV | Roof/0.025 | Medium | 90 | 0.081 | 950 | 0.9 | 0.7 | 0.7 | Gauvin and |
| | | rough | | | | | | | Vette (2020) |
| | Wall/0.05 | | | | | | | | |
| MC | Roof/0.025 | Medium | 599 | 0.069 | 6894 | 0.9 | 0.7 | 0.7 | |
| | | rough | | | | | | | |
| | Wall/0.05 | | | | | | | | |

LECA: Lightweight expanded clay aggregate;
EV: Expanded vermiculite;
MC: Mycelium composite.

TABLE 8
Representative cities selected from different
US climate zones for building energy modeling

| Zone | | | Representative |
|---|---|---|---|
| No. | Condition | Thermal criteria | city |
| 1 | Very hot, | 5000 < CDD10° C. | Miami, FL |
| 2 | Humid | 3500 < CDD10° C. ≤ | Phoenix, AZ |
| | Hot, dry | 5000 | |
| 3 | Warm, | CDD10° C. ≤ 2500 | San Francisco, |
| | | and HDD18° C. | CA |
| 4 | Marine | ≤2000 CDD10° C. ≤ | Albuquerque, |
| | Mixed, | 2500 and 2000 ≤ | NM |
| | dry | HDD18° C. ≤ 3000 | |
| 5 | Cool, dry | 3000 ≤ HDD18° C. ≤ | Salt Lake, UT |
| | | 4000 | |
| 6 | Cold, dry | 4000 ≤ HDD18° C. ≤ | Helena, MT |
| | | 5000 | |
| 7 | Very cold | 5000 < HDD18° C. ≤ | Duluth, MN |
| | | 7000 | |
| 8 | Subarctic | 7000 < HDD18° C. | Fairbanks, AK |

CDD: cooling degree day; HDD: heating degree day.

### The Effects of Different Thermal Insulation Materials on the Temperature Processes on Inner Roof Surface of Model Building Located in Albuquerque, NM

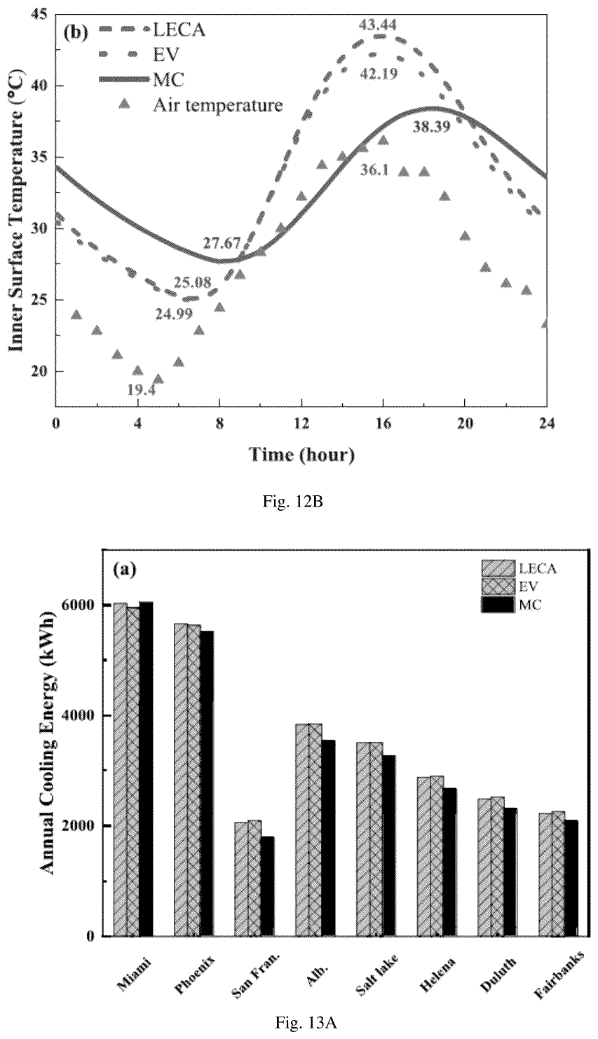

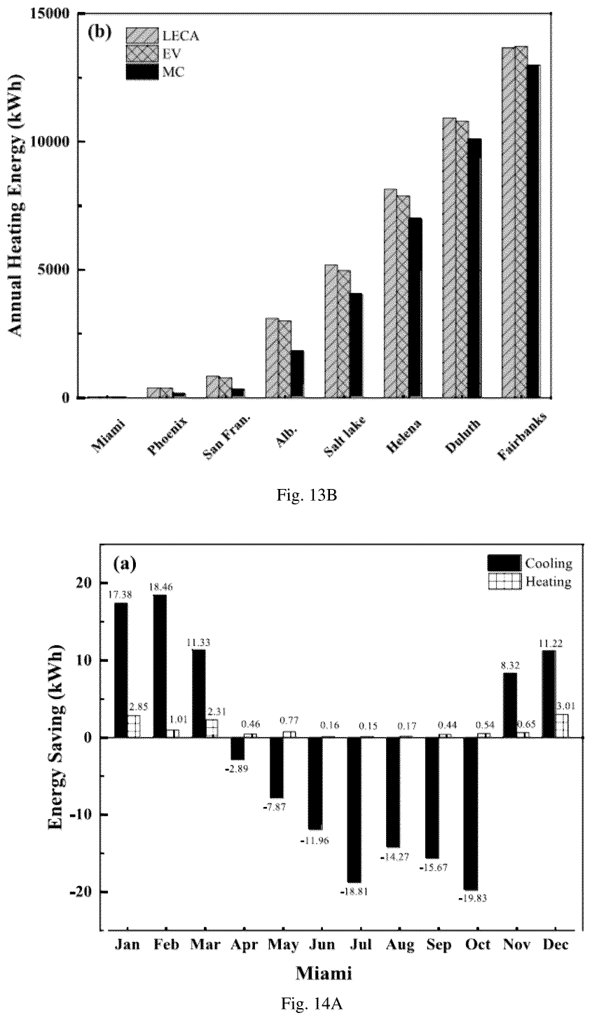

To observe the thermal insulation effects directly, the temperature on the inner roof surface in the buildings with mycelium-composite as well as conventional LECA, EV insulators were obtained and compared. The inner roof surface temperature affects heating and cooling energy consumption by applying thermal boundaries on the indoor environment. The HVAC system was automatically working and energy was consumed when the indoor temperature was changed out of the range from heating set-point temperature (20° C.) to cooling set-point temperature (26° C.). FIG. 12 presents the inner roof surface in the models with various insulators as well as the outside air temperature during a representative day in winter or summer. As shown in FIG. 12(A), the lowest outside air temperature was −5.6° C. in winter and the lowest temperature on the inner roof surface was modulated to 12.72° C., 13.16° C. and 15.52° C. by LECA, EV and mycelium-composite insulators respectively. The highest outdoor air temperature was 13.3° C. at around 4:00 p.m., and the maximum temperature of the inner roof surface was 31.38° C., 31.36° C., and 26.33° C. when using LECA, EV and mycelium-composite insulation layer respectively. The performances of insulation materials on a summer day were compared in FIG. 12(B). The lowest outdoor air temperature was 19.4° C. and the corresponding lowest inner roof surface temperature was 25.08° C., 24.99° C., and 27.67° C. respectively for LECA, EV and mycelium-composite insulating materials. Meanwhile, the highest inner roof surface temperature was 43.44° C., 42.19° C., and 38.39° C. for buildings with LECA, EV and mycelium-composite insulators respectively. The modulated temperature of the inner roof surface was higher than the highest air temperature (36.1° C.), due to absorption of solar radiation. For both winter and summer days, the use of mycelium composite insulation led to a smaller magnitude of indoor temperature fluctuations compared with conventional LECA or EV insulation materials. This indicates that the mycelium-composite had superior thermal insulation than conventional insulation and saved energy consumption in operation. The peak of the inner roof surface temperature lagged behind the peak of air temperature. The lagging effects were impacted by the heat storage and release of materials, and the lagging effect was more obvious with the increase of specific heat capacity. The peaks of inner roof surface temperature in the building with LECA and EV were located at similar positions since LECA and EV have the same specific heat capacity. The temperature changed slowly in the building with mycelium-composite due to its highest specific heat capacity.

### Effects of Mycelium Composite Thermal Insulation on the Annual Heating and Cooling Energy Consumption of Model Building Located in Different Climate Zones

The annual energy use for heating and cooling were compared for buildings with mycelium-composite insulators versus with LECA or EV insulators in different climate zones. The results of energy consumption for the cooling and heating based on EnergyPlus analyses are shown in FIGS. 13 (A) and (B).

The cooling energy consumption was less for buildings with mycelium-composite insulators in the climate Zone 2-8 compared to the buildings with LECA and EV insulators, as shown in FIG. 13 (A) and Table 9. The annual energy uses for cooling saved by 145.71 kWh, 278.40 kWh, 294.39 kWh, 241.05 kWh, 220.83 kWh, 176.69 kWh, as well as 135.00 kWh in comparison to LECA, and 117.87 kWh, 316.23 kWh, 297.61 kWh, 247.20 kWh, 245.93 kWh, 210.24 kWh, as well as 166.88 kWh in comparison to EV when using the mycelium-composite insulation in the building located in Phoenix, San Fran., Albuquerque, Salt Lake, Helena, Duluth and Anchorage respectively. The only exception was the annual cooling energy use for buildings with mycelium-composite insulators increased by 24.61 kWh and 103.31 kWh in very hot zone (Miami, FL).

Compared to its impacts on cooling energy use, mycelium-composite insulation had better performance on the reduction of heating energy consumption. The highest of total annual heating energy saved by the mycelium-composite insulation compared to LECA and EV occurred in the Albuquerque (1311.86 kWh and 1216.52 kWh), followed by Salt Lake (1159.59 kWh and 935.47 kWh), Helena (1164.51 kWh and 907.69 kWh), Duluth (808.37 kWh and 682.96 kWh), Fairbanks (672.26 kWh and 731.53 kWh), San Fran. (520.41 kWh and 472.38 kWh) and Phoenix (238.11 kWh and 231.96 kWh). However, only 12.52 kWh and 12.13 kWh of reduction of heating energy were found in the very hot region (i.e., Miami, FL).

Therefore, the building with mycelium-composite insulation consumed less energy for heating and cooling compared to that with conventional insulation materials (i.e., LECA and EV) in general. The only exception was the annual cooling energy in Miami, FL, which is under very hot climate condition. Moreover, the reduction of cooling and heating energy use was more significant in colder regions.

TABLE 9
Annual building cooling and heating energy saving by use of mycelium-composite
insulator compared with conventional insulator (LECA and EV)

| | | | Saved ACE | Saved ACE | Saved AHE MC | Saved AHE |
|---|---|---|---|---|---|---|
| Zone | | Representative | MC vs. | MC vs. EV | vs. LECA | MC vs. EV |
| No. | Condition | city | LECA (kWh) | (kWh) | (kWh) | (kWh) |
| 1 | Very hot, | Miami, FL | −24.61 | −103.31 | 12.52 | 12.13 |
| | humid | | | | | |
| 2 | Hot, dry | Phoenix, AZ | 145.71 | 117.87 | 238.11 | 231.96 |
| 3 | Warm, | San Francisco, | 278.40 | 316.23 | 520.41 | 472.38 |
| | marine | CA | | | | |
| 4 | Mixed, dry | Albuquerque, | 294.39 | 297.61 | 1311.86 | 1216.52 |
| | | NM | | | | |
| 5 | Cool, dry | Salt Lake, UT | 241.05 | 247.20 | 1159.59 | 935.47 |
| 6 | Cold, dry | Helena, MT | 220.83 | 245.93 | 1164.51 | 907.69 |
| 7 | Very cold | Duluth, MN | 176.69 | 210.24 | 808.37 | 682.96 |
| 8 | Subarctic | Fairbanks, AK | 135.00 | 166.88 | 672.26 | 731.53 |

MC: Mycelium Composite;
ACE: Annual Cooling Energy;
AHE: Annual Heating Energy.

### Monthly Energy Saving

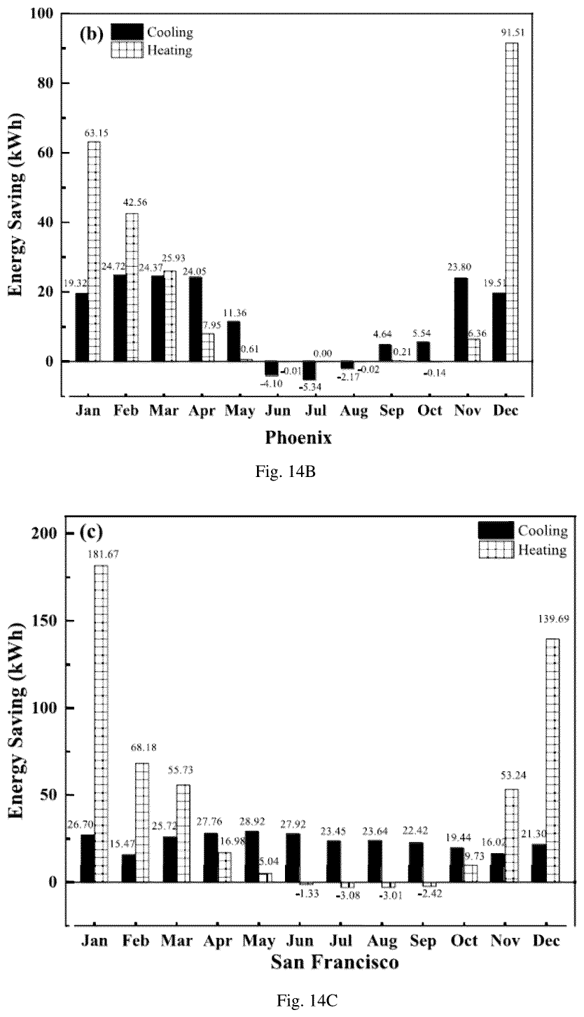

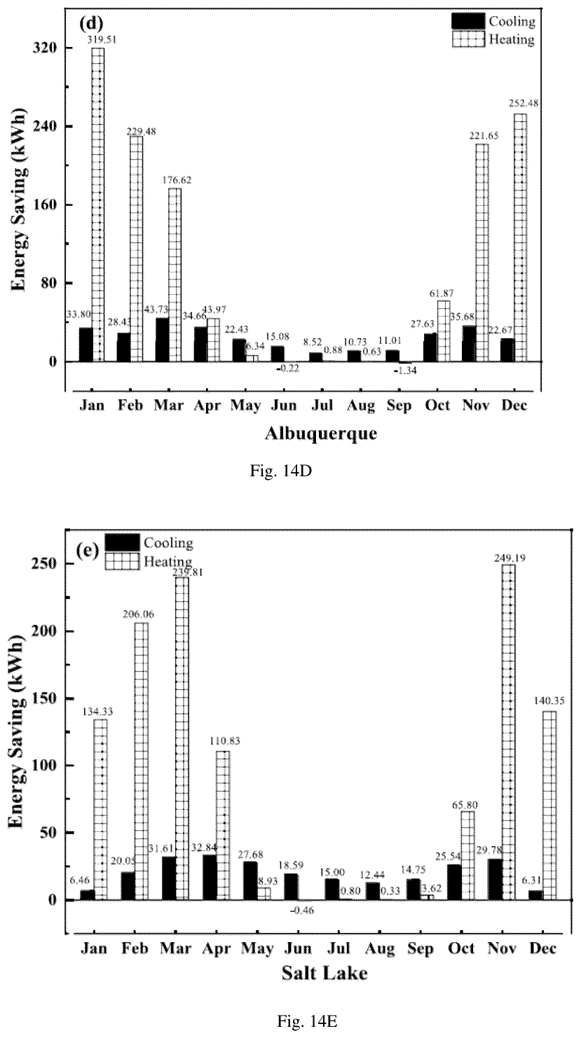

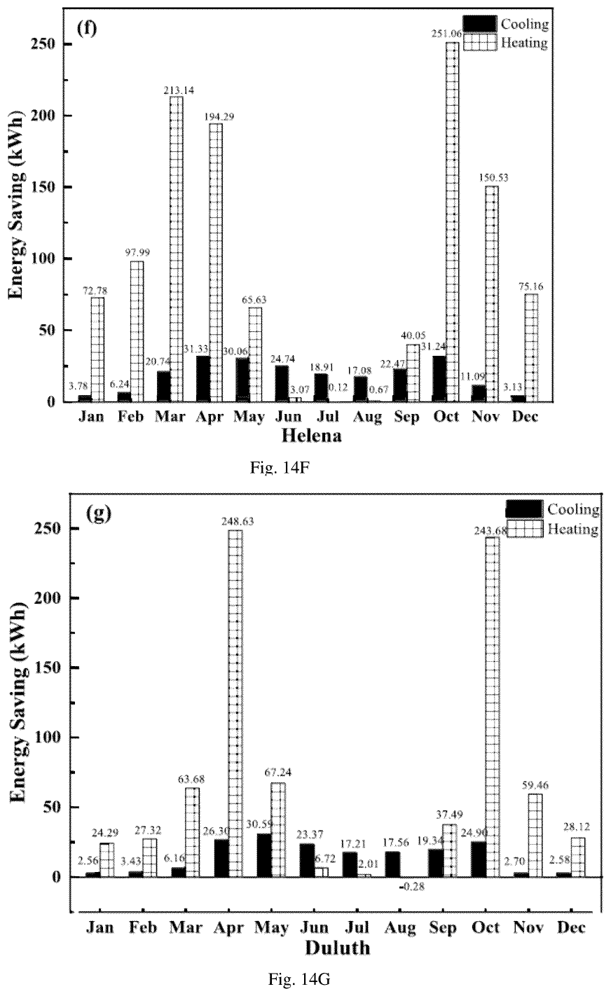

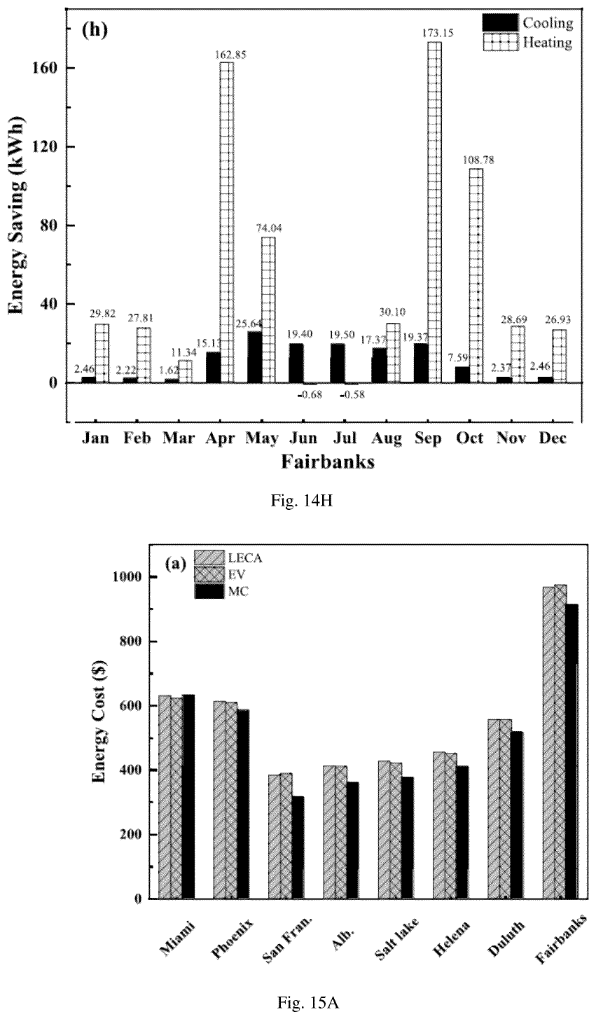

Monthly energy saving for cooling and heating by mycelium-composite insulation compared with a LECA insulator at different climate regions is shown in FIG. 14. In general, during the winter, the heating and cooling energy demands were decreased with the use of mycelium-composite insulation. However, the increase of energy used for the heating or cooling occurred in some locations during summer.

Although mycelium-composite insulation saved heating and cooling energy in winter, it increased the cooling energy use from April to October in Miami (FIG. 14 (A)). A similar result could be seen from the FIG. 14 (B), the energy for heating and cooling in the building with mycelium-composite insulation decreased as much as 91.51 kWh in December and 24.72 kWh in February, however, the monthly cooling energy increased by 4.1 kWh, 5.34 kWh and 2.17 kWh from June to August. For climate zones with cooler climate conditions (Zone 3-8), the heating and cooling energy used in the building with mycelium-composite decreased significantly in the winner; the heating and cooling energy consumption was similar or only slightly higher than that of the building with LECA insulator in the summer. The highest monthly cooling energy saving was 28.92 kWh, 43.73 kWh, 32.84 kWh, 31.33 kWh, 30.59 kWh and 25.64 kWh for the building located in San Fran., Albuquerque, Salt Lake, Helena, Duluth, and Fairbanks respectively; while the highest monthly heating energy saving of 181.67 kWh, 319.51 kWh, 249.19 kWh, 251.06 kWh, 248.63 kWh and 173.15 kWh is achieved respectively at these locations.

### Energy Cost and Equivalent Carbon Emission

The energy cost saving and the energy associated equivalent carbon emission of building models with various building insulation materials were investigated under different climate zones. The unit prices for electricity and natural gas were gained from residential prices provided by U.S. Energy Information Administration (Table 10) (Administration et al., 2019). The equivalent carbon dioxide emission for cooling and heating energy was estimated by multiplying the factors of 0.758 kgCO₂/kWh and 0.232 kgCO₂/kWh for electricity and natural gas (ANSI/ASHRAE/USGBC/IES, 2010).

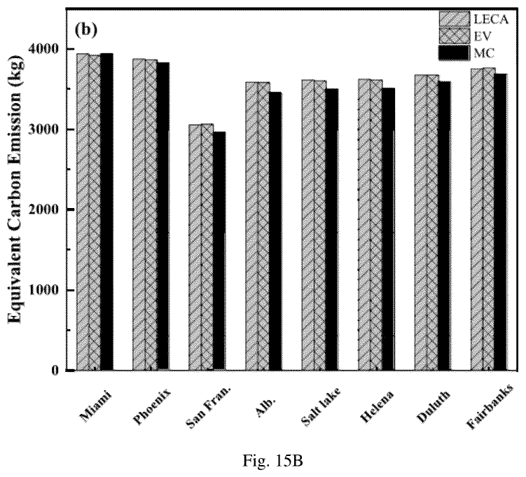

As shown in FIG. 15 (a) and Table 11, the mycelium-composite insulation reduced the annual energy cost of building operation for Zone 2-8 and slightly increased in Zone 1, which is the very hot climate region such as Miami, FL. When compared with LECA and EV, mycelium-composite insulation reduced the annual energy cost by $26.30 and $23.08, $70.03 and $74.30, $55.13 and $53.34, $50.82 and $45.35, $48.11 and $44.16, $40.50 and $40.51, $52.80 and $61.49 for building located in Phoenix, San Fran., Albuquerque, Salt Lake, Helena, Duluth and Fairbanks respectively.

TABLE 11
Summary of annual saving in building energy cost and
CO₂ emission reduction by the use of mycelium-composite
insulator compared with conventional insulator (LECA and EV)

| | | | | Reduced | Reduced |
|---|---|---|---|---|---|
| | | Saved | Saved | CO₂ | CO₂ |
| | | cost vs. | cost vs. | Emission | Emission |
| Zone | Representative | LECA | EV | vs. LECA | vs. EV |
| No. | city | ($) | ($) | (kg) | (kg) |
| 1 | Miami, FL | −1.64 | −9.89 | −5.09 | −23.44 |
| 2 | Phoenix, AZ | 26.30 | 23.08 | 46.19 | 39.39 |
| 3 | San Francisco, CA | 70.03 | 74.30 | 91.63 | 97.97 |
| 4 | Albuquerque, NM | 55.13 | 53.34 | 136.10 | 131.94 |
| 5 | Salt Lake, UT | 50.82 | 45.35 | 115.84 | 105.73 |
| 6 | Helena, MT | 48.11 | 44.16 | 111.38 | 104.00 |
| 7 | Duluth, MN | 40.50 | 40.51 | 82.76 | 84.12 |
| 8 | Fairbanks, AK | 52.80 | 61.49 | 66.05 | 76.52 |

FIG. 15(B) presents the carbon dioxide emission associated with operating the building in Climate Zone 1-8. Table 11 summarizes the reduction of the equivalent carbon dioxide emission by use of mycelium-composite insulators for the model building compared to LECA and EV insulators. The highest carbon emission reduction by the mycelium-composite insulation compared with LECA and EV insulation were about 136.10 kg and 131.94 kg, which is achieved in Albuquerque. This was followed by Salt Lake (115.84 kg reduction compared with LECA and 105.73 kg reduction compared with EV respectively), Helena (111.38 kg and 104.00 kg respectively), San Fran. (91.63 kg and 97.97 kg respectively), Duluth (82.76 kg and 84.12 kg respectively), Fairbanks (66.05 kg and 476.52 kg respectively) and Phoenix (46.19 kg and 39.39 kg respectively). The only exception is for Zone 1 in very hot zone (i.e., Miami, FL), where the annual CO₂ emission by building operation was increased (by 5.09 kg and 23.44 kg respectively by MC insulator compared with LECA or EV).

This example explored the potential of biologically growing mycelium composites as natural building insulation materials. The mycelium composite bricks were naturally grown with edible fungus, Pleurotus ostreatus, using rye berry grains as biodegradable substrates. Their moisture-related mechanical properties were investigated. Additionally, the energy performance of buildings with mycelium-composite insulation was analyzed with EnergyPlus and compared with traditional natural insulation materials (LECA and EV). The central conclusions include:

The analysis of the microstructure of the mycelium composite bricks indicated that fungal fibers grow and feed on rye berry grains, bonding them together to form a porous composite.

With an increase of density, composites presented an improvement of flexural strength under both low and middle levels of RH. However, under conditions of high RH, mycelium composite showed a similar bending behavior regardless of density levels. These composites' flexural strength was relatively lower than that of composites exposed to low and middle levels of RH. Regardless of the RHs, the mechanical strength of the mycelium composites is well above the existing requirements for building insulation materials.

The effects of mycelium-composite insulation on the energy performance of buildings located in different US climate zones were analyzed using EnergyPlus. Compared with LECA and EV insulation, the use of a mycelium-composite insulator reduced the inner surface temperature fluctuations of the roof by 7.39 and 7.85° C. respectively during a typical winter day and by 6.48 and 7.64° C. respectively during a typical summer day in Albuquerque, NM. The analyses indicated that the use of mycelium composite insulation saved more heating energy during winter than cooling energy during summer.

The mycelium-composite insulation reduced the annual energy cost of building operations for model buildings located in Zone 2-8, and also lowered their CO₂ emissions. The highest total annual carbon emissions reduction by mycelium composite insulation was achieved in Albuquerque, by 136.10 kg and 131.94 kg respectively compared with LECA and EV insulation. The only exception was for Zone 1, which is a very hot climate region including such places as Miami, FL, where uses of the mycelium composite insulation increased both the total energy usage and CO₂ emissions.

Overall, the mycelium-composite insulation material will achieve better energy performance than both the conventional natural insulators in most US climate zones, except in Zone 1, or very hot and humid climates. In addition to its energy performance, the material can be naturally grown and can potentially substitute petroleum-derived materials for a more sustainable development strategy.

### Experimental Procedure and Results

### Sample Preparations

The fungi spawn of Pleurotus ostreatus, which is free from injurious diseases and pests, was utilized in this study. The fungi spawn containing numerous hyphal vegetative tissue and spores was purchased from the Mushroom Spawn Laboratory at Pennsylvania State University. Composite samples were prepared by growing fungi spawn with rye berries.

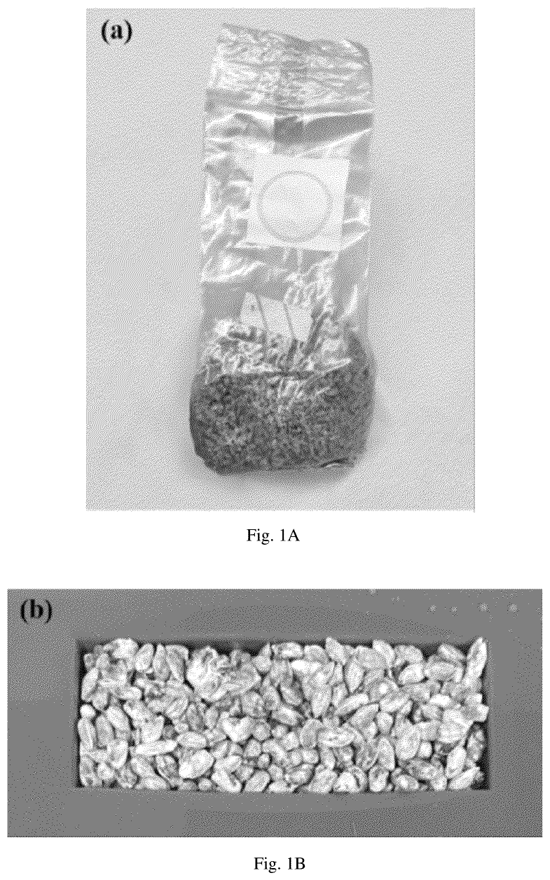

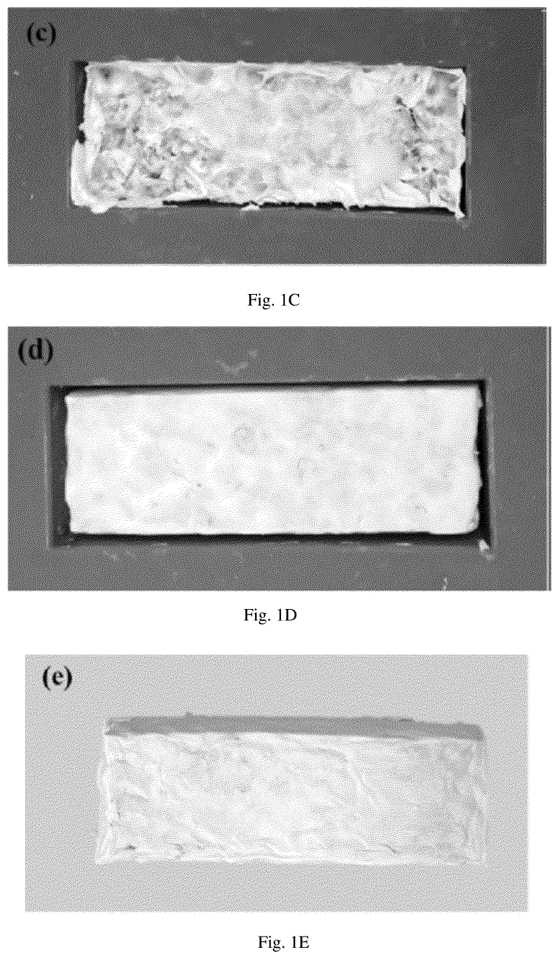

The procedure of samples preparation is demonstrated in FIG. 1. A filter patch bag with 454 g of rye berries was sterilized at 121° C. for 2.5 h by using saturated steam at 15 psi prior to use. The fungal spores germinated and fungal hyphae colonized into the feedstocks (rye berries), which was observed clearly by naked eyes after 3 days. After a 7-day incubation period, the mixture of fungi spawn and feedstocks were removed from bag and manually ground homogenized. 40 g, 42 g, and 45 g of myceliated feedstocks (produced from one bag) were transferred to sterilized silicone gel molds. Over the period, the hyphae bonded with grain particles and formed an increasingly tight network. The substrate was allowed to grow in the mold for four days, and it was flipped over for additional four days to minimize the errors generated due to sampling. The incubation environment for insulation bricks formed by fungal mycelium was controlled as 65% RH and 25° C. Then the bricks were placed in the oven for 24 h at 65° C. to kill any living microbes in the mycelium composites.

Fungal fibers took up nutrients from the feedstock by releasing enzymes that converted feedstock into breakdown products. The process generates a composite. Fungal mycelium became denser and more robust on the surface of composites when the mycelium migrated to the borders of the mold. The robust layer on the surface was also called fungal skin. Thus, the mycelium composites consisted of a core of colonized substrate, wrapped by a layer of fungal skin outside (FIG. 1).

### Microstructure Characterization

Helios NanoLab 650 was utilized to observe the morphology and microstructure of the produced mycelium composites. The applied high voltage was 5 kV and the working distance was 4-5 mm for the characterization with scanning electron microscopy. The voltage and current used in energy dispersive X-ray spectrometry were set as 15 kV and 1.6 nA respectively.

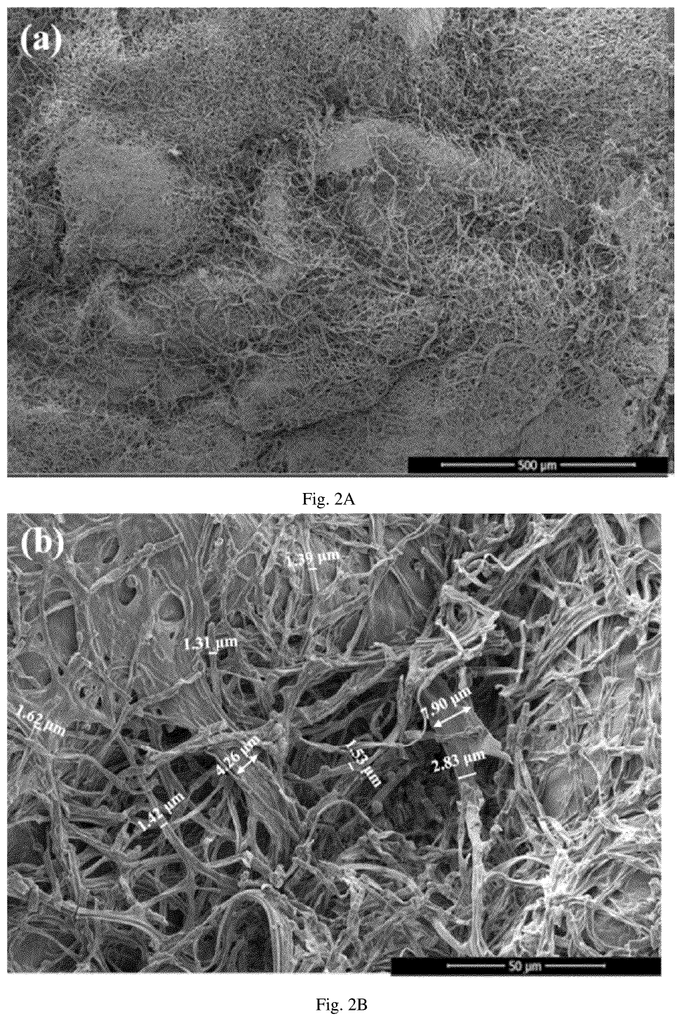

After mixing fungi spawn with feedstocks, fungi developed hyphae that penetrated into grains and released enzymes. The colonization of fungal hyphae was initiated on feedstocks. From SEM images, fungal fibers attached to the surface of the feedstocks, forming a fibrous network by branching and entanglement of hyphae (FIG. 2(A)). Branching is the interaction and crosslinking; entanglements are the physical overlap between hyphae. Both branching and entanglements increase the strength of composites. The formed composites are multi-scale materials that contain different phases. Images were taken at various separate sites and representative images are provided in FIG. 2. The feedstocks grains are on the order of 2-5 mm. The diameter of single fungal fibers obtained from analysis of its microstructure range between 1.31-1.62 m. The fungal hyphae bonded together and coalesced to generate bridge structures between adjacent grains. The width of the bridge ranged from 2.83 m to 7.90 m. The matrix phase of the formed composite is a random fiber network. This porous structure improves the thermal insulation performance of the composites.

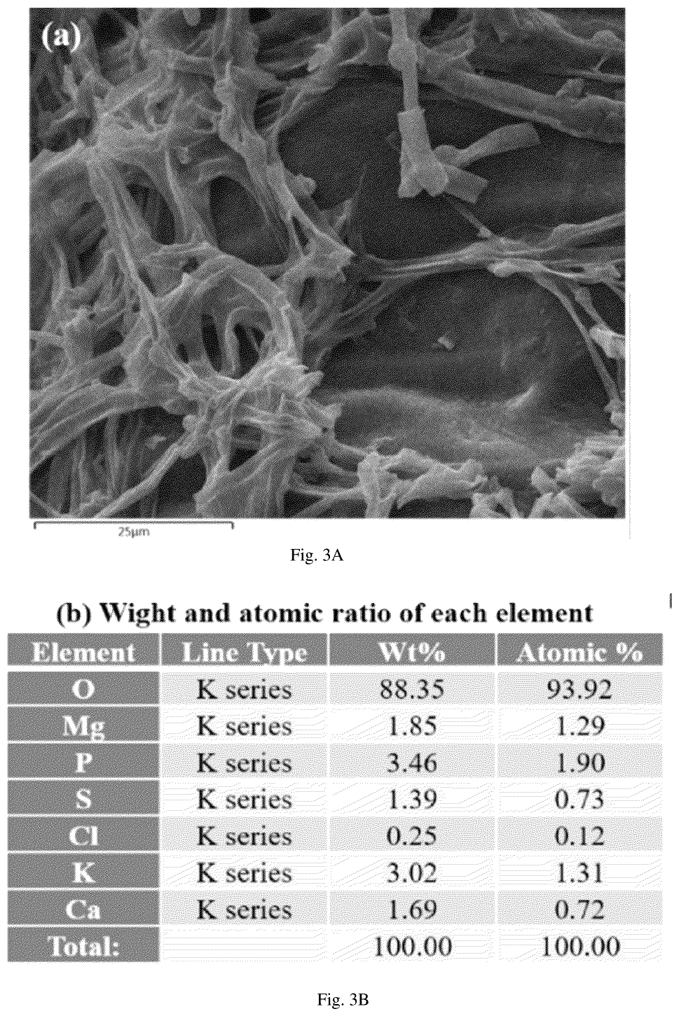

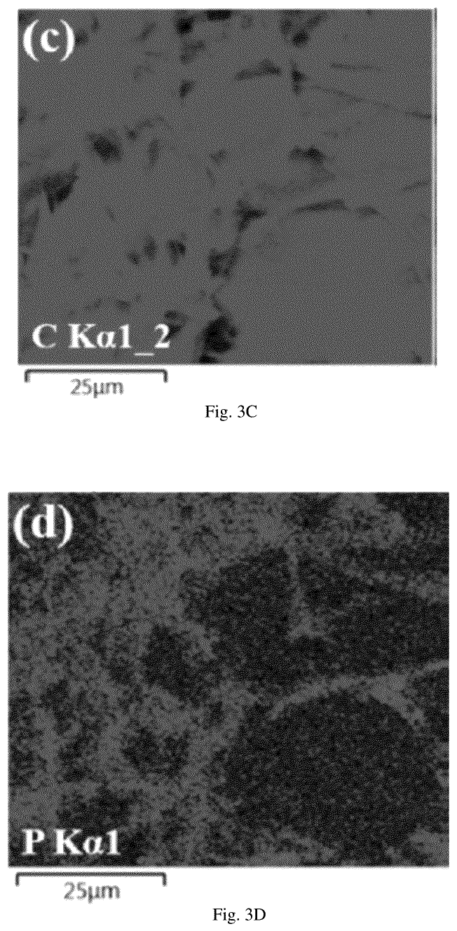

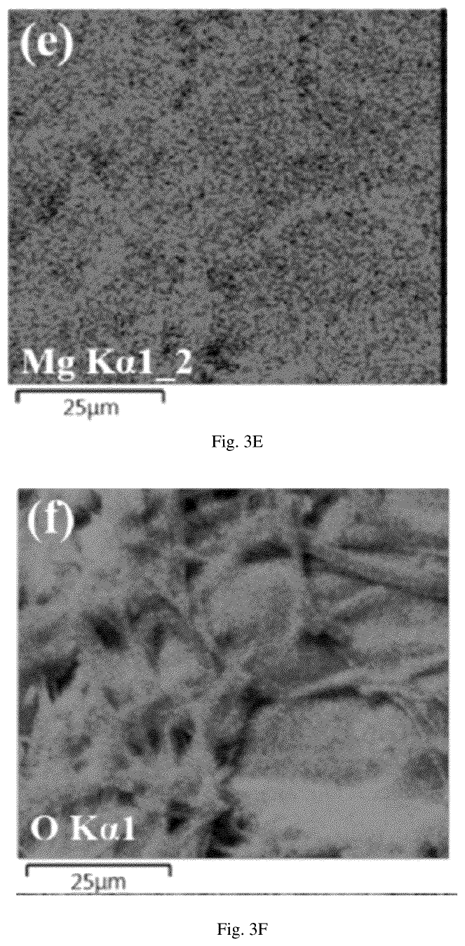

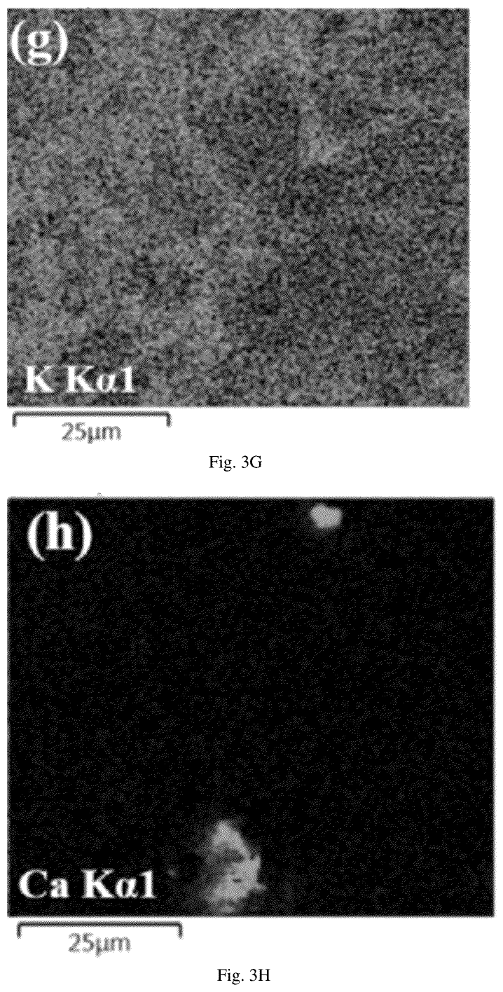

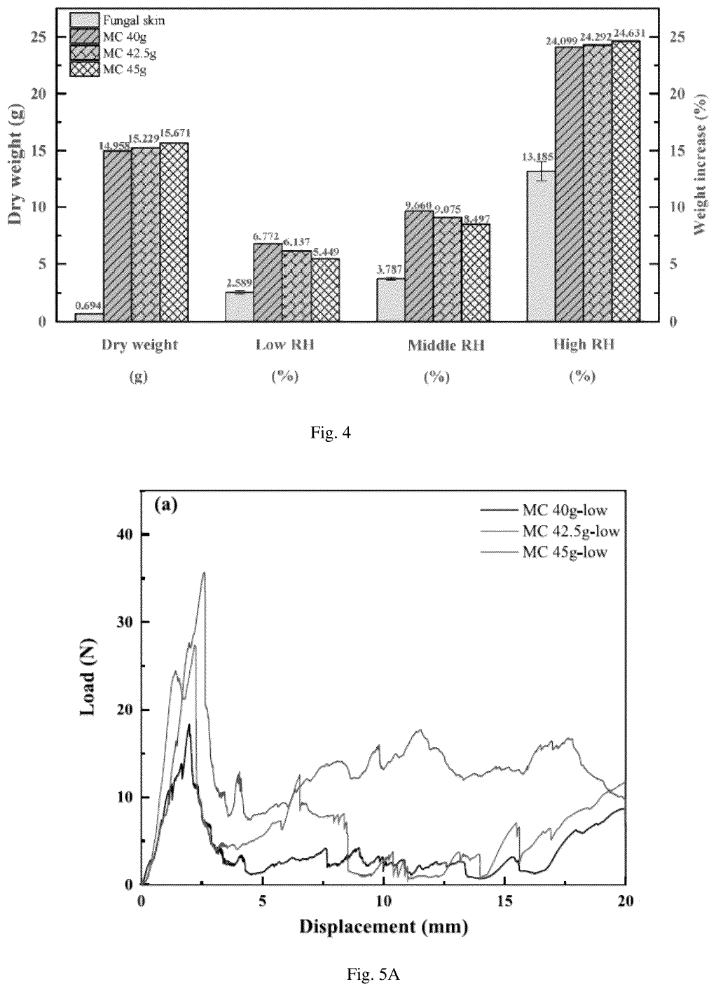

EDS analyses were conducted to map the main chemical elements contained in mycelium composites. The element mapping (FIG. 3(C-H)) demonstrates that mycelium grown from grains contained Carbon, Oxygen, Phosphorus, Potassium, and Magnesium. Additionally, several Calcium-rich areas were observed on the surface of the mycelium (i.e., FIG. 3 (H)). The EDS analyses detected the presence of phosphorus in the composites. It is widely reported that phosphorus promotes the formation of char, improving fire retardancy. Materials containing phosphorus are flame-retardant. The presence of phosphorus in mycelium composites is beneficial for their application in building construction (see FIG. 4).

### Moisture Behavior

The contact angle measurement of fungal skin was performed with a goniometer (KSV CAM200 Instruments Ltd) to evaluate its water repellency. Water was dispersed at a constant speed and the contact profile of water on fungal skin was captured by a high-resolution camera. The temperature and the relative humidity were controlled to be 23° C. and 49%, respectively. Five different locations with dimensions of 1 cm×1 cm were chosen from fungal skin. The average contact angle is 116.35° with a standard error of 0.958°, which indicates that fungal skin is highly hydrophobic.

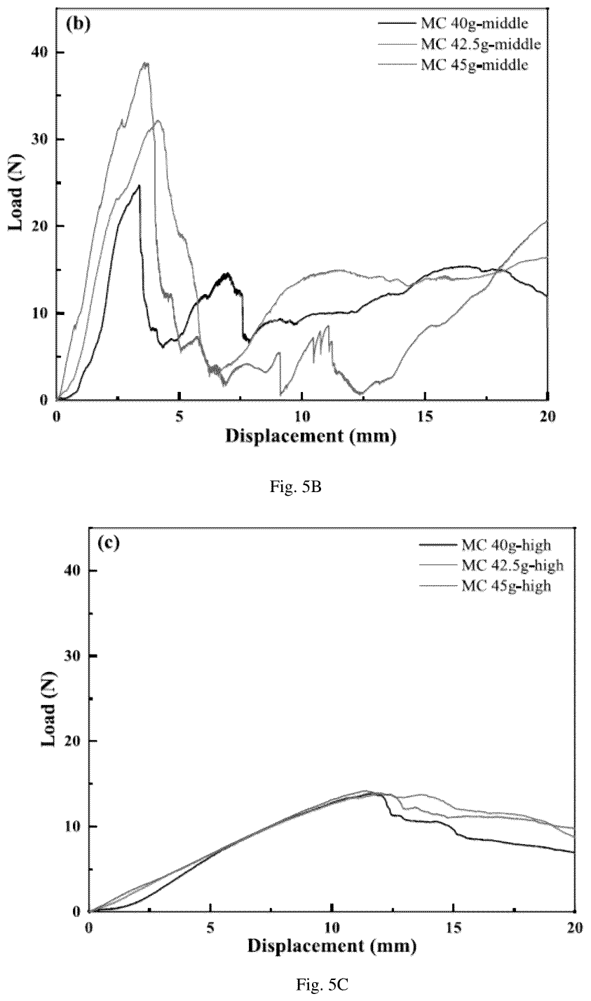

The impacts of ambient RH on the mechanical property of mycelium composites were investigated under low, middle and high relative humidity (RH) levels adapted from (Yang and Marr, 2012). Mycelium composites were stored in moisture chambers with RH of 23%, 54%, and 98% for 72 h until they reached constant masses. The initial and final masses were used to determine the capacity of water absorption in the composites. The results show that when exposed to low and middle levels of RH, composites grown with more substrates had a smaller weight increase and their water bottom surface. When the tensile stress exceeded the tensile strength of the composite, micro cracks gradually appeared on the bottom surface. It indicates the mycelium composites are prone to fracture under the tension state. Micro cracks progressed to macro cracks, developing from the bottom to the top surface. The bearing load had a sudden drop after reaching the peak load, which was associated with the increase of deformation of the composite. Cracks gradually propagated on the side of the sample, and composites retained a low level of load-bearing capacity due to the relative lower density of the mycelium in the core of composites. The fungal skin showed higher resistance to external load. Therefore, the sample retained an increased load capacity when the crack propagated near the top surface before the sample fully fractured, as shown in FIGS. 5(A) and (B). The flexural strength of mycelium composites ranged from 172 to 335 kPa, and 232-364 kPa after exposure to low and middle RH, respectively. With the increase of substrate contained in the composite, it gained a higher flexural strength. Compared to other non-pressed mycelium-composite materials that present flexural strength varying from 50 to 290 kPa (Appels et al., 2019), the mycelium composites fabricated in this study are able to bear higher bending loads. In addition, the composites grown with 40 g, 42.5 g, and 45 g of substrate exposed to middle RH had a 34.88%, 17.97%, and 8.66% larger bear capacity compared to the corresponding samples exposed to low RH.

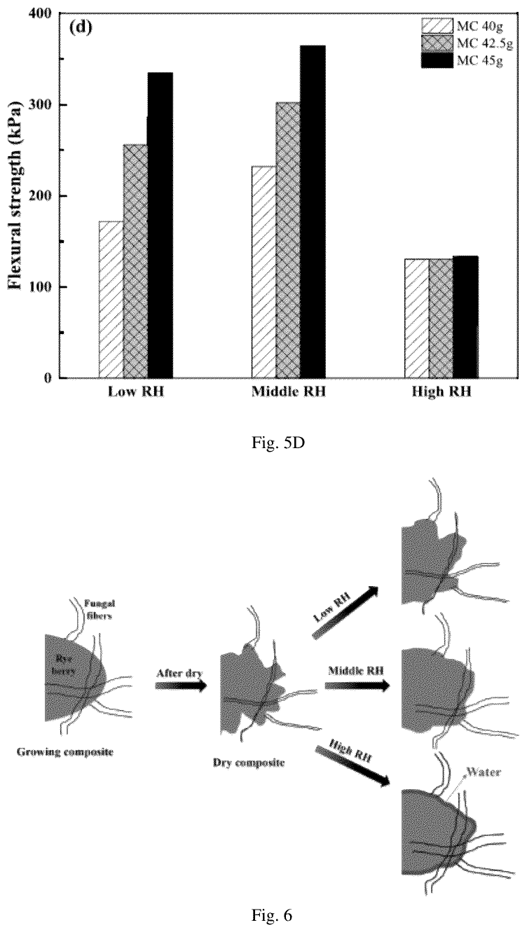

FIG. 6 illustrates the changes that occurred in composites when exposed to low, middle, and high RHs. Compared to fungal skin, rye berry grains were sensitive to humidity. Hydrophobic fungal skin was water-repellent and protected grains from interacting with water when exposed to low RH. Dry berry grains were brittle and the connections of mycelium on the shriveled grains were susceptible to breakage, leading to the low bearing capacity. The bonding effect between mycelium and grains gradually recovered due to moisturisation when the composites are exposed to the middle level of RH. However, exposing mycelium composites to high level of RH led to grains surrounded by a layer of absorbed water when exposed to high RH, which led to quick failure of interaction between mycelium and grains. Under such conditions, the density of the substrate had limited influence on the flexural strength of mycelium composite, as shown in the load-deformation curves in FIG. 5(C).

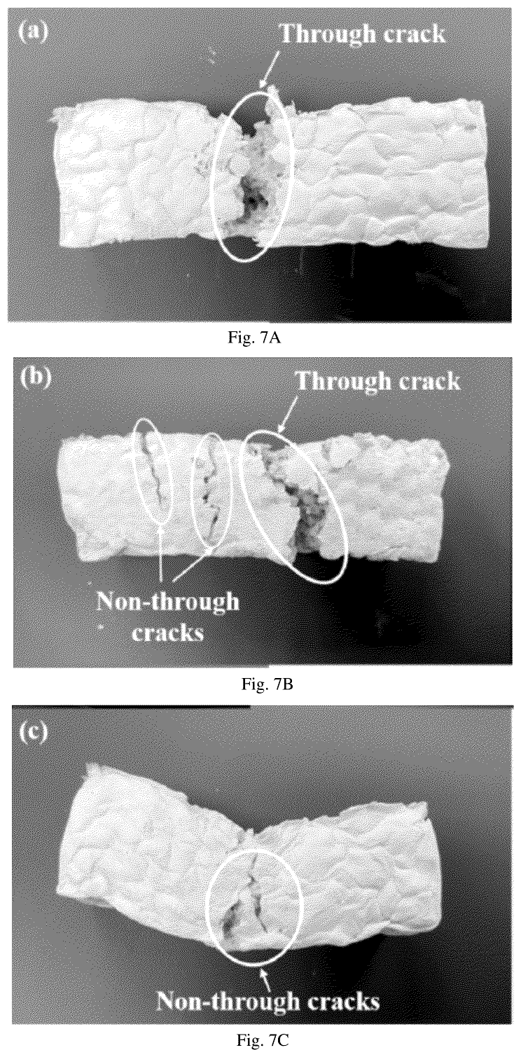

Nine groups of mycelium composites demonstrated three different failure modes under different levels of ambient RHs. Composites with different densities presented a consistent failure mode at the same RH level. The typical failure modes of the mycelium composites were captured after the bending test, as shown in FIG. 7. Under low RH, dry composites were brittle and only one macro crack clearly displays on the surface of composite. Spallation occurs when the crack initiates from the bottom to the top (FIG. 7A). After being exposed to the middle level of RH, the mycelium composites displayed a multiple-cracking phenome-non before fully fractured (FIG. 7B), which was similar to cement composites reinforced by fibers. Increasing RH level enhanced the water content in the feedstocks, resulting in stronger interaction between mycelium and the feedstocks. The stronger bridging effect of fungal fibers promotes the redistribution of stress, leading to the visible multiple cracking. Surprisingly, under high level of RH=98%, density of substrate did not remarkably affect the load-displacement behaviors of mycelium composites, and the curves resembled each other. This is attributed to the fact that both fibers and rye grains were saturated and softened when exposed to RH=98%. They were ductile and able to bear large deformation. Besides, only non-through cracks appeared at the bottom of the composite after a large deformation (FIG. 7C). The water layer absorbed by grains undermined the bonding effect between fibers and grains, which contributed to the low bearing capacity in the composite with high water content. The breakage of fungal skin and loss of bonding in the partial area presented in the composites after exposure to high level of RHs.

Although insulation materials are not the main load-bearing component in a building, its mechanical property must meet the requirements to avoid damage during transporting and construction. The minimum requirement of flexural strength for commercial insulation (EPS) is 70 kPa according to ASTM C578-18. Overall, mycelium composites with 45 g substrate (MC 45 g) generated a porous structure with dense and robust mycelium. The porous structure is beneficial to thermal insulation performance. The flexural strength of MC 45 g under three levels of RH was measured as 335 kPa, 364 kPa, and 133 kPa, all of which are significantly higher than the requirements. To further quantify the performance of mycelium composite insulation, the following study focused on the thermal properties characterization and building energy performance analyses for mycelium composite grown with 45 g substrate.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A mycelium composite material comprising:
a fungi generated mycelium fiber matrix; and
a biofiller dispersed within the matrix, wherein the biofiller provides a structural element and nutrition source for the fungi and the mycelium fibers bind the biofiller.

2. The mycelium composite material of claim 1, wherein the biofiller is an organic filler, an inorganic filler, or a combination thereof.

3. The mycelium composite material of claim 1, further including a core of fungi colonized substrate encased by a water-repellant fungi skin, wherein the core and skin include a mycelium fiber network

4. An insulation material comprising a mycelium composite material of claim 1.

5. The insulation material of claim 4, being configured for insulating a building or building envelope.

6. The insulation material of claim 4, wherein the biofiller is an organic filler, an inorganic filler, or a combination thereof.

7. The insulation material of claim 4, further including a core of fungi colonized substrate encased by a water-repellant fungi skin, wherein the core and skin include a mycelium fiber network

8. A building envelope comprising:
a mycelium composite insulation provided on or within the building envelope, wherein the mycelium composite insulation includes a fungi generated porous mycelium fiber matrix and a biofiller dispersed within the matrix, wherein the biofiller provides a structural element and nutrition source for the fungi and the mycelium fibers bind the biofiller.

9. The building envelope of claim 8, wherein the biofiller is an organic filler, an inorganic filler, or a combination thereof.

10. The building envelope of claim 8, wherein mycelium composite material includes a core of fungi colonized substrate encased by a water-repellant fungi skin, wherein the core and skin include a mycelium fiber network.

11. A method of forming a building material, the method comprising:
cultivating a mycelium generating fungus on a biomaterial feedstock;
grinding the cultivated fungus and feedstock after generation of fungal spores and/or fungal hyphae colonization into the feedstock;
transferring the ground fungi and feedstock to a mold;
cultivating the ground fungi and feedstock in the mold such that a mycelium composite is formed with a core of fungi colonized substrate encased by a water-repellant fungi skin; and
providing nutrition and/or biofiller to tailor fungi properties.

12. The method of claim 11, wherein the biofiller includes a source of silica in the nutrition source to enhance fire resistance of the mycelium composite.

13. The method of claim 11, further comprising heating the mycelium composite to kill any living microbes in the mycelium composite.

14. The method of claim 11, further comprising pressure molding the mycelium composite to enhance mechanical strength.

15. The method of claim 11, further comprising providing the mycelium composite insulation on or within a building envelope.
